(19) United States
(12) Patent Application Publication
(10) Pub. No.: US 2021/0358581 A1
(43) Pub. Date: Nov. 18, 2021

(54) SECURED VALIDATION SYSTEM

(71) Applicant: VC, Inc., Cleveland, OH (US)

(72) Inventors: Bernardo Moreno, Westlake, OH (US); Shane M. Bigelow, Gates Mills, OH (US); Bo J. Shim, Avon, OH (US); Michael D. Ornelas, Draper, UT (US); Pengyu Chen, Cottonwood Heights, UT (US); Kevin Steffensen, Midvale, UT (US); Branden Russell, Salt Lake City, UT (US)

(21) Appl. No.: 17/318,798

(22) Filed: May 12, 2021

Related U.S. Application Data
(60) Provisional application No. 63/069,933, filed on Aug. 25, 2020.
(60) Provisional application No. 63/023,677, filed on May 12, 2020.

(51) Int. Cl. G16H 10/60; H04L 29/08; H04L 9/06; G06F 21/62; H04L 9/30

(52) U.S. Cl. CPC G16H 10/60; H04L 67/306; H04L 2209/38; G06F 21/6245; H04L 9/3066; H04L 9/0643

(57) ABSTRACT

Methods for securely validating a test are provided. In one aspect, the method includes receiving an enrollment request from a user device associated with an individual. The method includes generating a profile associated with the individual and storing the profile on a blockchain network, wherein the profile comprises profile data. The method includes receiving a test result associated with a test of the individual and updating the profile with the test result. The method includes determining whether a facility associated with the test result is an approved certifying facility. The method includes creating, responsive to determining that the facility is one of the approved certifying facilities, a unique code associated with the individual, wherein the unique code comprises an indication of the test result. The method includes transmitting the unique code to the user device associated with the individual. Systems and machine-readable media are also provided.

### CROSS-REFERENCE TO RELATED APPLICATION

The present application claims the benefit of priority under 35 U.S.C. § 119 from U.S. Provisional Patent Application Ser. No. 63/023,677 entitled “Secured Validation System,” filed on May 12, 2020, and from U.S. Provisional Patent Application Ser. No. 63/069,933 entitled “Secured Validation System,” filed on Aug. 25, 2020, the disclosures of which are hereby incorporated by reference in its entirety for all purposes.

### TECHNICAL FIELD

The present disclosure generally relates to validation systems, and more specifically relates to secured validation systems.

### BACKGROUND

In some instances, data is stored at a central location such as a central server. While centrally storing the data provides some general security of the data, it is not convenient for a third party to access that data. Third party access to such data is made even more difficult when the software of the central server and the software of the third party are different.

The description provided in the background section should not be assumed to be prior art merely because it is mentioned in or associated with the background section. The background section may include information that describes one or more aspects of the subject technology.

### SUMMARY

According to certain aspects of the present disclosure, a method for securely validating a test is provided. The method includes receiving an enrollment request from a user device associated with an individual. The method includes generating a profile associated with the individual and storing the profile on a blockchain network, wherein the profile comprises profile data. The method includes receiving a test result associated with a test of the individual and updating the profile with the test result. The method includes determining whether a facility associated with the test result is an approved certifying facility. The method includes creating, responsive to determining that the facility is one of the approved certifying facilities, a unique code associated with the individual, wherein the unique code comprises an indication of the test result. The method includes transmitting the unique code to the user device associated with the individual.

According to other aspects of the present disclosure, a system for securely validating a test is provided. The system includes a memory comprising instructions and a processor configured to execute the instructions which, when executed, cause the processor to receive an enrollment request from a user device associated with an individual. The processor is configured to execute the instructions which, when executed, cause the processor to generate a profile associated with the individual, wherein the profile comprises profile data. The processor is configured to execute the instructions which, when executed, cause the processor to store the profile on a blockchain network. The processor is configured to execute the instructions which, when executed, cause the processor to receive a test result associated with a test of the individual. The processor is configured to execute the instructions which, when executed, cause the processor to append the profile of the individual with the test result. The processor is configured to execute the instructions which, when executed, cause the processor to determine whether a facility associated with the test result is approved certifying facility. The processor is configured to execute the instructions which, when executed, cause the processor to create, responsive to determining that the facility is one of the approved certifying facilities, a unique code associated with the individual, wherein the unique code comprises an indication of the test result. The processor is configured to execute the instructions which, when executed, cause the processor to create, responsive to transmit the unique code to the user device associated with the individual.

According to other aspects of the present disclosure, a non-transitory machine-readable storage medium comprising machine-readable instructions for causing a processor to execute a method is provided. The method includes receiving an enrollment request from a user device associated with an individual. The method includes generating a profile associated with the individual, wherein the profile comprises profile data. The method includes storing the profile on a blockchain network. The method includes receiving a test result associated with a test of the individual. The method includes updating the profile of the individual with the test result. The method includes determining whether a facility associated with the test result is approved certifying facility. The method includes creating, responsive to determining that the facility is one of the approved certifying facilities, a unique code associated with the individual, wherein the unique code comprises an indication of the test result. The method includes transmitting the unique code to the user device associated with the individual.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram schematically illustrating an exemplary secured validation system, according to certain aspects of the disclosure.

FIG. 2 is a block diagram illustrating the example server and devices from FIG. 1, according to certain aspects of the disclosure.

FIG. 3 illustrates an example process for using the example server and devices of FIG. 2, according to certain aspects of the disclosure.

FIGS. 4A-4E are example illustrations associated with the example process of FIG. 3.

FIG. 5 is a block diagram illustrating an example computer system with which the server and devices of FIG. 2 can be implemented.

FIG. 6 is a diagram schematically illustrating alternative aspects of the exemplary secured validation system, according to certain aspects of the disclosure.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

General Overview

The disclosed systems and methods provide a confidential, secure, and immutable validation system. For example, the validation system can consolidate and validate health status information, although other validation systems are certainly within the scope of this disclosure. The validation system can be based on a blockchain network. The blockchain network can be a public blockchain, a private blockchain, a consortium blockchain, or a hybrid blockchain. The disclosed system is a peer-to-peer (P2P) system and implements a database on a blockchain network. In such a manner, the blockchain network of the disclosed systems confidentially and securely manages the data stored in the database.

Continuing with the example of validating health status information, the disclosed system receives enrollment requests from a plurality of individuals. After an individual is enrolled properly and has a test performed at a testing facility, the disclosed system receives a corresponding test result of the individual based on the test performed and verifies the test result. Subsequent to verifying that the test result of the individual matches or came from a verified testing facility, the disclosed system issues a unique code associated with the individual. The unique code associated with the individual includes an indication of the test result. The disclosed system transmits the unique code to a mobile device associated with the individual. The unique code can be displayed on the mobile device associated with the individual and presented and/or scanned at an establishment that requires a specific indication of the test result associated with the unique code in order for the individual to be allowed entry into the establishment. As a non-limiting example, the individual may be trying to enter the establishment, such as an airport, and presents the unique code via the mobile device. The airport scans the unique code and identifies the indication of the test result, for example, as “Immunity Present,” which meets the requirement guidelines for the airport such that the individual is allowed to enter the airport.

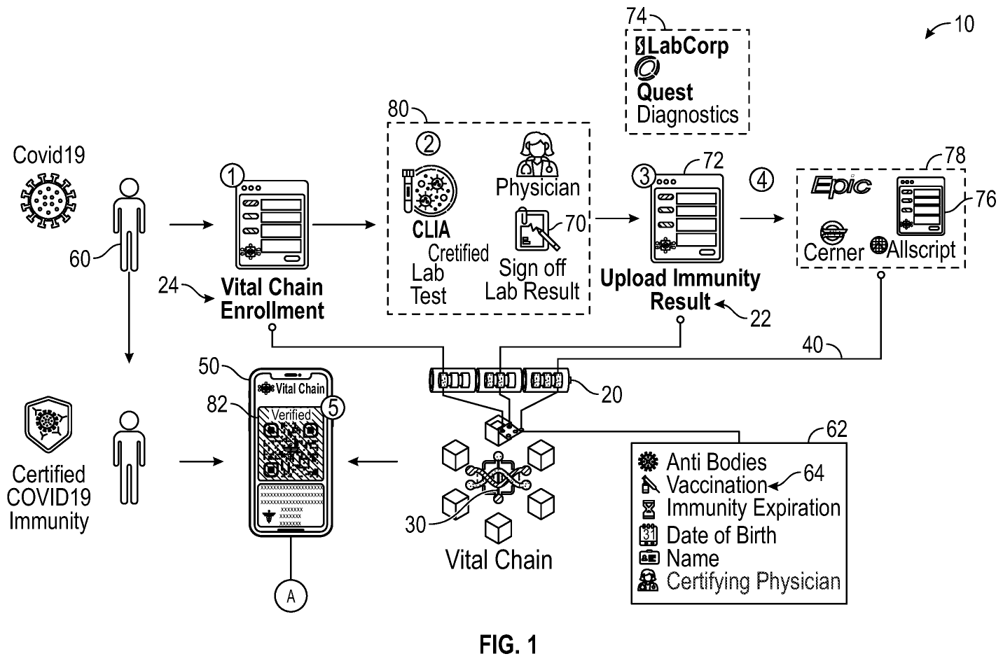

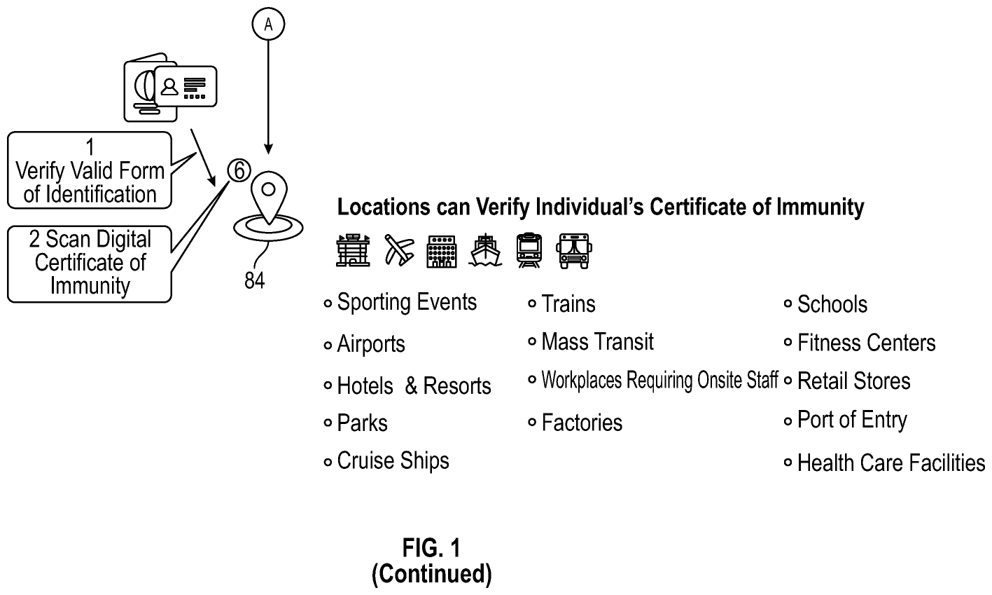

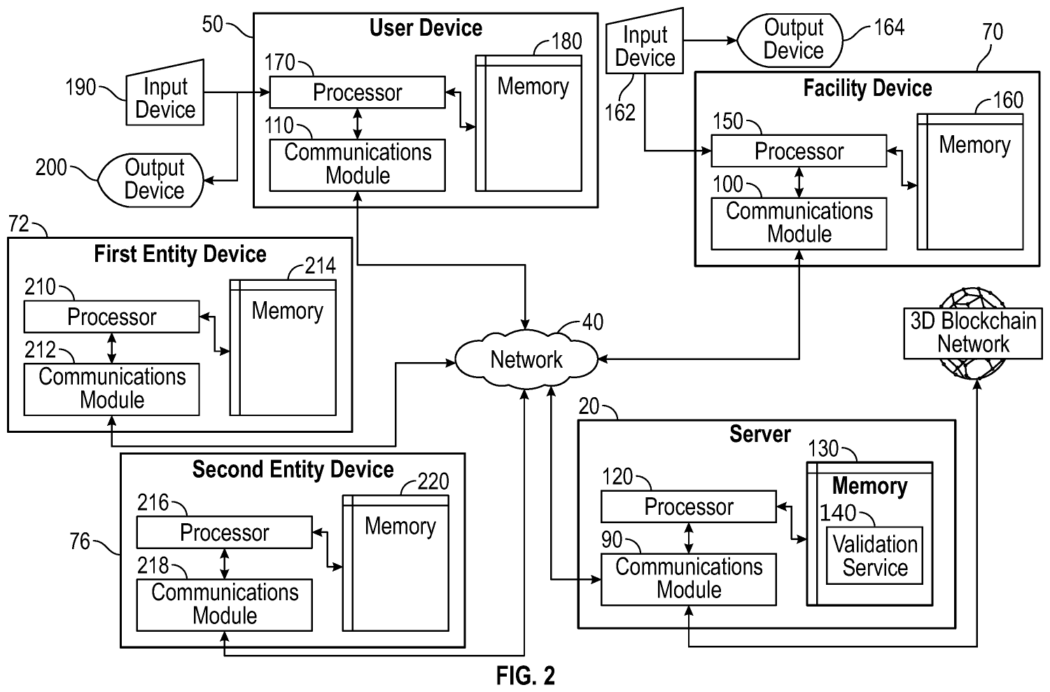

FIG. 1 illustrates an example diagram schematically illustrating a secured validation system 10 for securely validating a test result. The validation system 10 includes a validation server 20 in communication with a blockchain network 30. The validation server 20 is in communication, over a network 40, with at least one user device 50, such as a mobile device associated with an individual 60, with at least one facility device 70, such as a computer associated with a facility 80, and with at least one first entity device 72, such as a computer, associated with a first entity 74. The facility 80 is any facility where a test is conducted such as, but not limited to, a physician office, a hospital, a clinic, a school, a university, a certified lab, such as, for example, a Clinical Laboratory Improvement Amendments (CLIA) certified laboratory, and other such well-known testing facilities. The first entity 74 can be a lab system entity such as, but not limited to, for example, Quest, LabCorp, and other lab system entities well-known in the industry. In some aspects, the validation server 20 is also in communication, over the network 40, with at least one second entity device 76, such as a computer, associated with a second entity 78. The second entity 78 can be a health system entity such as, but not limited to, for example, a hospital, Epic, Cerner, Allscripts, and other health system entities well-known in the industry. The validation server 20 is configured to host a validation service 140 (see FIG. 2). For purposes of load balancing, multiple servers can host the validation service 140.

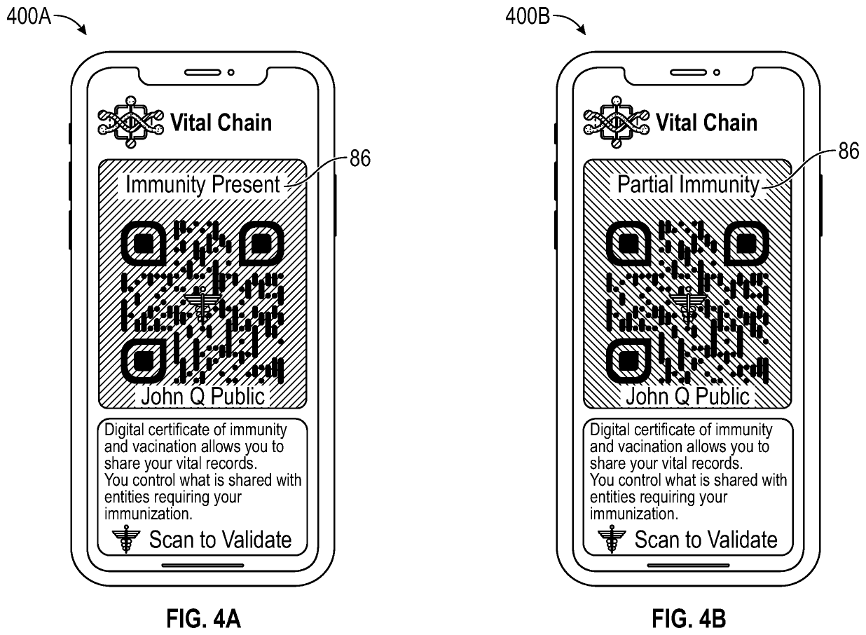

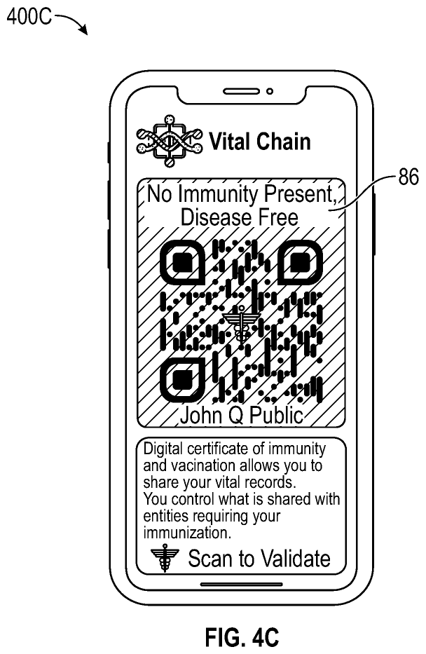

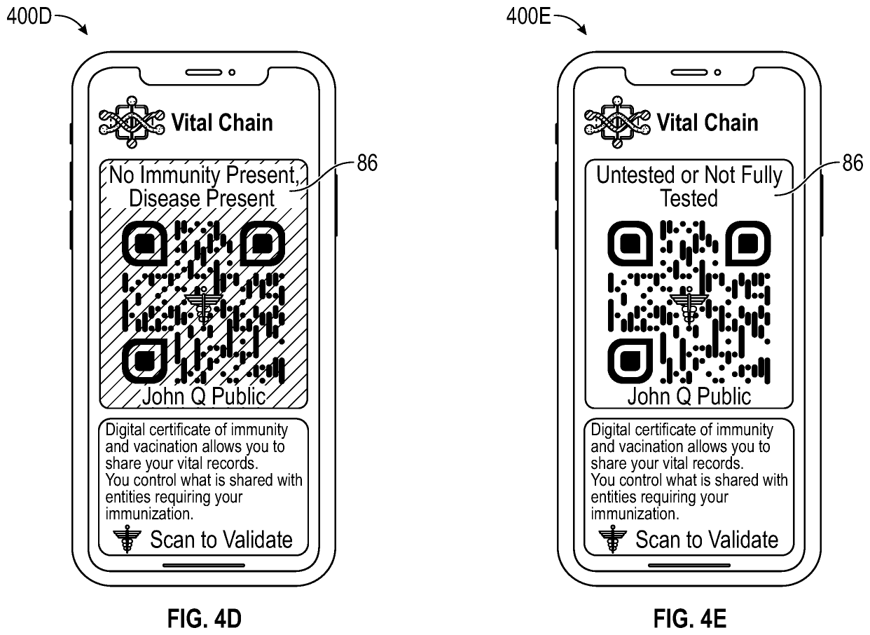

In certain aspects, the validation server 20 receives test results, such as a test result 22, associated with enrolled individuals, such as the individual 60, and validates or verifies the test results. For example, the validation service 140 hosted on the validation server 20 registers the individual 60 in response to an enrollment request 24 via the user device 50 associated with the individual 60. As part of registering the individual 60, for example, the validation service 140 generates a profile 62 associated with the individual 60 and stores the profile 62 on the blockchain network 40. The profile 62 includes profile data 64, which can include, but is not limited to, a name, a date of birth, an indication 86 (see FIGS. 4A-4E) of the test result 22, antibody indicators, vaccination indicators, certifying physician or facility, and other data. After the individual 60 completes the test, the at least one first entity device 72 of the first entity 74 receives the test from the facility 80 via the facility device 70 and generates a corresponding test result 22. The validation service 140 then receives the corresponding test result 22 from the first entity 74 via the at least one first entity device 72. With the test result 22 being certified by the facility 80, the validation server 20 then determines whether the facility 80 is an approved certifying facility. In response to determining that the facility 80 is an approved certifying facility, the validation service 140 creates a unique code 82 associated with the individual 60 that includes the indication 86 of the test result 22. The validation service 140 then transmits the unique code 82 to the user device 50 so that the individual 60 can present the unique code 82 to an establishment 84. The establishment 84 can then scan the unique code 82 on the user device 50 to determine whether the individual is allowed to enter the establishment 84 based on the indication 86 of the test result 22 identified by the unique code 82.

In certain alternative aspects, instead of receiving the test result 22 from the first entity 74, the validation service 140 receives the test result 22 from the second entity 78 via the at least one second entity device 76. In such aspects, the second entity 78, instead of the validation server 140, determines whether the facility 80 is an approved certifying facility.

The validation server 20 can be any device having an appropriate processor, memory, and communications capability for hosting the validation service 140. The at least one user device 50 and the at least one facility device 70 to which the validation server 20 is connected over the network 40 can be, for example, desktop computers, mobile computers, tablet computers (e.g., including e-book readers), mobile devices (e.g., a smartphone or PDA), set top boxes (e.g., for a television), video game consoles, or any other devices having appropriate processor, memory, and communications capabilities. In certain aspects, the validation server 20 can be a cloud computing server of an infrastructure-as-a-service (IaaS) and be able to support a platform-as-a-service (PaaS) and software-as-a-service (SaaS) services.

The network 40 can include, for example, any one or more of a personal area network (PAN), a local area network (LAN), a campus area network (CAN), a metropolitan area network (MAN), a wide area network (WAN), a broadband network (BBN), the Internet, and the like. Further, the network 40 can include, but is not limited to, any one or more of the following network topologies, including a bus network, a star network, a ring network, a mesh network, a star-bus network, tree or hierarchical network, and the like.

FIG. 2 is a block diagram illustrating the validation server 20, the facility device 70, and the mobile device 50 in the validation system 10 of FIG. 1, according to certain aspects of the disclosure.

The validation server 20, the facility device 70, and the mobile device 50 are connected over the network 40 via respective communication modules 90, 100, 110. The communications modules 90, 100, 110 are configured to interface with the network 40 to send and receive information, such as data, requests, responses, and commands to other devices on the network 40. The communications modules 90, 100, 110 can be for example, modems or Ethernet cards.

The validation server 20 includes a processor 120, the communications module 90, and a memory 130 that includes the validation service 140. The processor 120 of the validation server 20 is configured to execute instructions, such as instructions physically coded into the processor 120, instructions received from software in memory 130, or a combination of both. The validation server 20 is also in communication with the blockchain network 30. For example, the processor 120 of the validation server 20 executes instructions from the validation service 140 causing the processor 120 to receive the enrollment request 24 from the user device 50 associated with the individual 60. The processor 120 of the validation server 20 executes instructions from the validation service 140 causing the processor 120 to generate, in response to receiving the enrollment request 24, the profile 62 associated with the individual 60 with the profile data 64. The processor 120 of the validation server 20 executes instructions from the validation service 140 causing the processor 120 to store the profile 62 on the blockchain network 40.

The processor 120 of the validation server 20 executes instructions from the validation service 140 causing the processor 120 to receive the test result 22 from the facility device 70 associated with the facility 80. In some implementations, the processor 120 of the validation server 20 executes instructions from the validation service 140 causing the processor 120 to receive the test result 22 from the user device 50. The processor 120 of the validation server 20 executes instructions from the validation service 140 causing the processor 120 to append the test result 22 to the profile 62 of the individual 60. The processor 120 of the validation server 20 executes instructions from the validation service 140 causing the processor 120 to determine whether the facility 80, from which the test result 22 was received, is an approved certifying facility. The processor 120 of the validation server 20 executes instructions from the validation service 140 causing the processor 120 to create, in response to determine the facility 80 is one of the approved certifying facilities, the unique code 82 associated with the individual 60 that includes the indication 86 of the test result 22. The processor 120 of the validation server 20 executes instructions from the validation service 140 causing the processor 120 to transmit the unique code 82 to the user device 50 associated with the individual 60.

The facility device 70 includes a processor 150, the communications module 100, and a memory 160. The facility device 70 also includes an input device 162, such as a keyboard or mouse, and an output device 164, such as a display. The processor 150 of the facility device 70 is configured to execute instructions, such as instructions physically coded into the processor 150, instructions received from software in memory 160, or a combination of both. For example, the processor 150 of the facility device 70 executes instructions to transmit the test results 22 to the validation server 20.

The mobile device 50 includes a processor 170, the communications module 110, and a memory 180. The mobile device 50 also includes an input device 190, such as a screen interface, and an output device 200, such as a display. The processor 170 of the mobile device 50 is configured to execute instructions, such as instructions physically coded into the processor 170, instructions received from software in memory 180, or a combination of both. For example, the processor 170 of the mobile device 50 executes instructions to transmit the enrollment request 24 to the validation server 20. The processor 170 of the mobile device 50 executes instructions to receive the unique code 82 from the validation server 20.

The first entity device 72 includes a processor 210, a communications module 212, and a memory 214. The processor 210 of the first entity device 72 is configured to execute instructions, such as instructions physically coded into the processor 210, instructions received from software in memory 214, or a combination of both. For example, the processor 210 of the first entity device 72 executes instructions to receive the test from the facility device 70. In certain aspects, the processor 210 of the first entity device 72 executes instructions to transmit the test result 22 to the validation server 20 or to the second entity device 76.

The second entity device 76 includes a processor 216, a communications module 218, and a memory 220. The processor 216 of the second entity device 76 is configured to execute instructions, such as instructions physically coded into the processor 216, instructions received from software in memory 220, or a combination of both. For example, the processor 216 of the second entity device 76 executes instructions to determine whether the facility 80 is an approved certifying facility. The processor 216 of the second entity device 76 executes instructions to transmit the test result 22 to the validation server 20.

The techniques described herein may be implemented as method(s) that are performed by physical computing device(s); as one or more non-transitory computer-readable storage media storing instructions which, when executed by computing device(s), cause performance of the method(s); or, as physical computing device(s) that are specially configured with a combination of hardware and software that causes performance of the method(s).

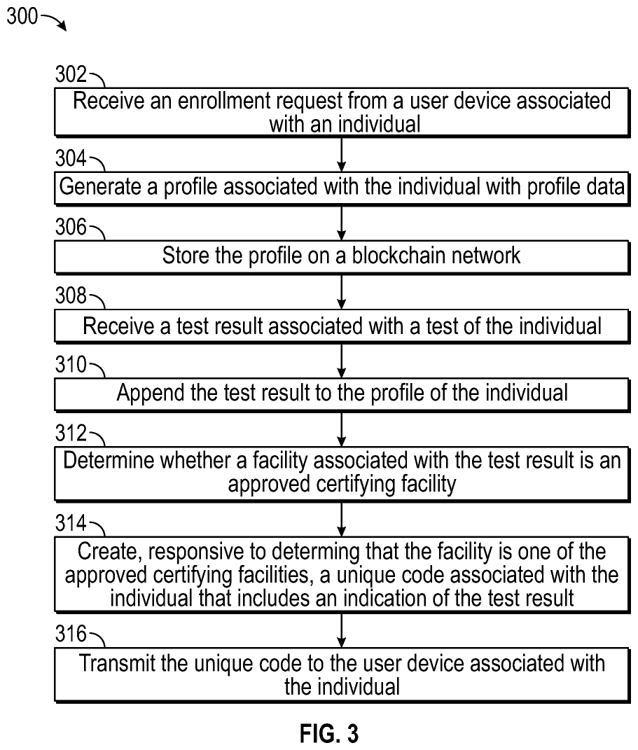

FIG. 3 illustrates an example process 300 for securely validating a test result using the example validation server 20 of FIG. 2. While FIG. 3 is described with reference to FIG. 2, it should be noted that the process steps of FIG. 3 may be performed by other systems.

The process begins by proceeding to step 302 when the processor 120 of the validation server 20 receives the enrollment request 24 from the user device 50 associated with the individual 60. As depicted in step 304, in response to receiving the enrollment request 24, the processor 120 of the validation server 20 generates the profile 62 associated with the individual 60 with the profile data 64. After the profile 62 is generated, the processor 120 of the validation server 20 stores the profile 62 on the blockchain network 30, as depicted at step 306. Subsequent to the individual 60 having the test be performed at the facility 80, the processor 120 of the validation server 20 receives the test result 22 associated with the test of the individual 60, as depicted in step 308. In certain aspects, the processor 120 of the validation server 20 receives the test result 22 from the first entity device 72 associated with the first entity 74. In some other aspects, the processor 120 of the validation server 20 receives the test result 22 from the second entity device 76 associated with the second entity 78.

At step 310, the processor 120 of the validation server 20 appends the test result 22 to the profile 62 of the individual 60. As depicted in step 312, the processor 120 of the validation server 20 determines whether the facility 80 associated with the test result 22 is an approved certifying facility. The processor 120 of the validation server 20 creates, responsive to determining that the facility 80 is one of the approved certifying facilities, the unique code 82 associated with the individual 60 that includes the indication 86 of the test result 22, as illustrated at step 314. At step 316, the processor 120 of the validation server 20 transmits the unique code 82 to the user device 50 associated with the individual 60. The unique code 82 can then be displayed on the user device 50 and presented to an establishment 84 for scanning to determine whether the unique code 82 includes the indication 86 of the test result 22 that meets the criteria to allow the individual 60 to enter the establishment 84.

FIG. 3 sets forth example process 300 for securely validating a test result using the example validation server 20, the blockchain network 30, the user device 50, and the facility device 70 of FIG. 2. An example will now be described using the example process 300 of FIG. 3 with reference to FIG. 1. The process 300 begins by proceeding to step 302, where the processor 120 of the validation server 20 receives the enrollment request 24 from the individual 60 who, for example, is going to get tested for an antibody or screening test. As an example, the individual 60 may be getting an antibody test to determine whether the individual has an immunity to coronavirus such as, but not limited to, COVID-19. It should be understood that antibody or screening tests to determine whether an individual has an immunity to other viruses are also within the scope of this disclosure. Moving to step 304, the processor 120 of the validation server 20 generates, in response to receiving the enrollment request 24, the profile 62 associated with the individual 60 with the profile data 64. The profile data 64 can include data such as, but not limited to, the name of the individual 60, the birth date of the individual 60, the indication 86 (see FIGS. 4A-4E) of the test result 22, antibody indicators, vaccination indicators, certifying physician or facility, and other data. Once the profile 62 is generated for the individual 60, as depicted in step 306, the processor 120 of the validation server 20 stores the profile 62 on the blockchain network 40. By storing such information on the blockchain network 40, the validation system 10 provides a secure and immutable record of the profile 62.

With the individual 60 enrolled, the individual 60 can then go to the facility 80 to get tested for immunity to a virus, such as COVID-19. The facility 80 can be any appropriate healthcare testing facility and the like. In some aspects, the facility 80 can be any facility where a test is conducted such as, but not limited to, a physician office, a hospital, a clinic, a school, a university, a certified lab, such as, for example, a Clinical Laboratory Improvement Amendments (CLIA) certified laboratory, and other such well-known testing facilities. After the facility 80 completes the test of the individual 60, the facility 80 can transmit, via the facility device 70, the test of the individual 60 to the first entity device 72 for generating the corresponding test result 22. Once the processor 120 of the validation server 20 receives the test result 22 of the individual 60 from the first entity device 72 of the first entity 74, the processor 120 appends the rest result 22 to the profile 62 of the individual 60, as depicted step 310. Upon updating the profile 62, as illustrated at step 312, the processor 120 of the validation server 20 determines whether the facility 80 associated with the test result 22 is an approved certifying facility.

At step 314, responsive to determining that the facility 80 is one of the approved certifying facilities, the processor 120 of the validation server 20 creates a unique code 82 associated with the individual 60 that includes the indication 86 of the test result 22. The unique code 82 can be any type of bar code or other well-known code in the industry. Example screenshots 400A, 400B, 400C, 400D, 400E of the unique code 82 is shown in FIGS. 4A-4E, respectively. In certain aspects, the unique code 82 can be color coded according to various results of the test result 22. For example, as depicted in FIG. 4A, the screenshot 400A of the unique code 82 can be green when the indication 86 of the test result 22 corresponds to “Immunity Present.” In similar examples, the screenshot 400B of the unique code 82, as depicted in FIG. 4B, can be blue when the indication 86 of the test result 22 corresponds to “Partial Immunity”; the screenshot 400C of the unique code 82, as depicted in FIG. 4C, can be yellow when the indication 86 of the test result 22 corresponds to “No Immunity Present, Disease Free”; the screenshot 400D of the unique code 82, as depicted in FIG. 4D, can be red when the indication 86 of the test result 22 corresponds to “No Immunity Present, Disease Present”; and the screenshot 400E of the unique code 82, as depicted in FIG. 4E, can be white when the indication 86 of the test result 22 corresponds to “Untested or Not Fully Tested.” While specific colors have been used as examples, it should be understood that any color can be used in such aspects. In certain aspects, the unique code 82 has an expiration time that is driven by the medical community's advice on how long it is valid and, once the expiration time expires, the unique code 82 reverts back to the unique code 82 depicted in FIG. 4E (e.g., white). In certain aspects, the name of the individual is included on the unique code 82. In such aspects, the name of the individual on the unique code 82 will help facilitate verification processes when a valid identification of the individual is required by the facility 80.

At step 314, the processor 120 of the validation server 20 transmits the unique code 82 to the user device 50 associated with the individual 60. With the unique code 82 on the user device 50, the individual 60 may wish to go to the establishment 84 which requires guests or employees to have taken a test and have test results that meet the criteria to allow the individual 60 to enter the establishment 84. For example, the individual 60 may wish to go to the establishment 84, such as a sporting event, and presents the unique code 82 on the user device 50 for the establishment 84 to scan. In this example, the unique code 82 is the screenshot 400A such that the indication 86 meets the criteria and the individual 60 is allowed to enter the establishment 84. In other scenarios, the facility 84 may only require the unique code 82 to include the indication 86 of the test result 22 corresponding to the screenshot 400B to allow entrance. While the establishment 84 is described as a sporting event in the above example, it should be understood that the establishment 84 can be any public or private entity establishment or transport vehicle such as, but not limited to, airports, hotels, resorts, parks, cruise ships, trains, mass transit vehicles, workplaces, factories, schools, fitness centers, retail stores, port of entries, health care facilities, and other well-known entities.

While the example process 300 illustrates an example aspect of the disclosed technology, it should be understood that additional and alternative processes are also within the scope of the present disclosure. For example, in some aspects, the processor 120 of the validation server 20 receives the enrollment request 24 from the user device 50 associated with the individual 60. In response to receiving the enrollment request 24, the processor 120 of the validation server 20 generates the profile 62 associated with the individual 60 with the profile data 64 as well as the unique code 82 associated with the individual 60, which can be updated later based on various criteria related to subsequent test results as described in more detail below. After the profile 62 and the unique code 82 is generated, the processor 120 of the validation server 20 stores the profile 62 and the unique code 82 on the blockchain network 30. Subsequent to the individual 60 having the test be performed at the facility 80, the processor 120 of the validation server 20 receives the test result 22 associated with the test of the individual 60. In certain aspects, the processor 120 of the validation server 20 receives the test result 22 from the first entity device 72 associated with the first entity 74. In some other aspects, the processor 120 of the validation server 20 receives the test result 22 from the second entity device 76 associated with the second entity 78.

In such aspects, the processor 120 of the validation server 20 appends the test result 22 to the profile 62 of the individual 60. The processor 120 of the validation server 20 can then determine whether the facility 80 associated with the test is an approved certifying facility. The processor 120 of the validation server 20 can also determine whether the test result 22 complies with predetermined requirements. In certain aspects, the predetermined requirements can include, but is not limited to, business rules regarding the validity of the stored test results 22 at the point of use, specific requirements such as Endpoint Service Location (ESL) requirements (e.g., such as requirements associated with the establishment 84), and other similar requirements well-known in the industry. The processor 120 of the validation server 20 updates, responsive to determining that the facility 80 is one of the approved certifying facilities and determining that the test result 22 complies with the predetermined business rules regarding the validity of the stored test results 22 at the point of use, the unique code 82 associated with the individual 60 to include the appropriate indication 86 of the test result 22. The processor 120 of the validation server 20 then transmits the unique code 82 to the user device 50 associated with the individual 60. The unique code 82 can then be displayed on the user device 50 and presented to an establishment 84 for scanning to determine whether the unique code 82 includes the indication 86 of the test result 22 that meets the criteria to allow the individual 60 to enter the establishment 84.

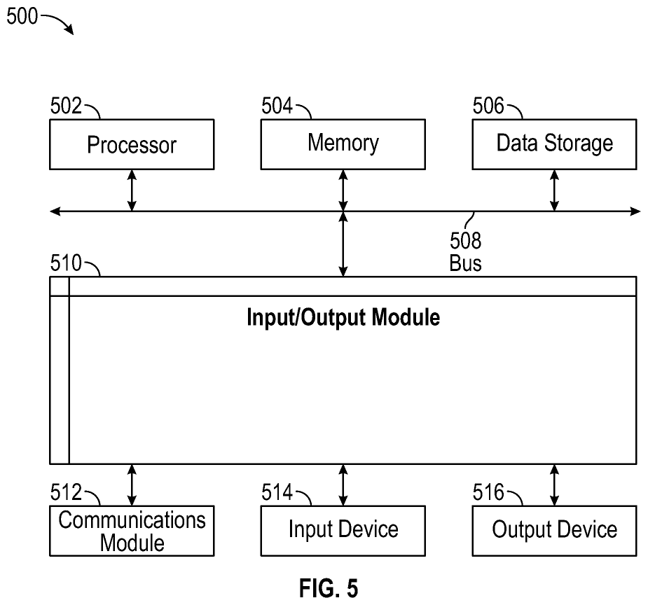

FIG. 5 is a block diagram illustrating an example computer system 500 with which the validation server 20, the user device 50, and the facility device 70 of FIG. 2 can be implemented. In certain aspects, the computer system 500 may be implemented using hardware or a combination of software and hardware, either in a dedicated server, or integrated into another entity, or distributed across multiple entities.

Computer system 500 (e.g., the validation server 20, the user device 50, the facility device 70) includes a bus 508 or other communication mechanism for communicating information, and a processor 502 (e.g., the processor 120, 150, 170) coupled with bus 508 for processing information. According to one aspect, the computer system 500 can be a cloud computing server of an IaaS that is able to support PaaS and SaaS services.

Computer system 500 can include, in addition to hardware, code that creates an execution environment for the computer program in question, e.g., code that constitutes processor firmware, a protocol stack, a database management system, an operating system, or a combination of one or more of them stored in an included memory 504 (e.g., memory 130, 160, 180), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 508 for storing information and instructions to be executed by processor 502. The processor 502 and the memory 504 can be supplemented by, or incorporated in, special purpose logic circuitry.

The instructions may be stored in the memory 504 and implemented in one or more computer program products, e.g., one or more modules of computer program instructions encoded on a computer readable medium for execution by, or to control the operation of, the computer system 500.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 500 further includes a data storage device 506 such as a magnetic disk or optical disk, coupled to bus 508 for storing information and instructions. Computer system 500 may be coupled via input/output module 510 to various devices (e.g., the input device 162, 190, the output device 164, 200). The input/output module 510 can be any input/output module. Example input/output modules 510 include data ports such as USB ports. In addition, input/output module 510 may be provided in communication with processor 502, so as to enable near area communication of computer system 500 with other devices. The input/output module 510 may provide, for example, for wired communication in some implementations, or for wireless communication in other implementations, and multiple interfaces may also be used. The input/output module 510 is configured to connect to a communications module 512. Example communications modules 512 (e.g., the communications module 90, 100, 110) include networking interface cards, such as Ethernet cards and modems.

In certain aspects, the input/output module 510 is configured to connect to a plurality of devices, such as an input device 514 (e.g., the input device 162, 190) and/or an output device 516 (e.g., the output device 164, 200). Example input devices 514 include a keyboard and a pointing device, e.g., a mouse or a trackball, by which a user can provide input to the computer system 500. Other kinds of input devices 514 can be used to provide for interaction with a user as well, such as a tactile input device, visual input device, audio input device, or brain-computer interface device.

According to one aspect of the present disclosure, the validation server 20, the user device 50, and the facility device 70 can be implemented using a computer system 500 in response to processor 502 executing one or more sequences of one or more instructions contained in memory 504. Such instructions may be read into memory 504 from another machine-readable medium, such as data storage device 506. Execution of the sequences of instructions contained in main memory 504 causes processor 502 to perform the process steps described herein. One or more processors in a multi-processing arrangement may also be employed to execute the sequences of instructions contained in memory 504. Processor 502 may process the executable instructions and/or data structures by remotely accessing the computer program product, for example by downloading the executable instructions and/or data structures from a remote server through communications module 512 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 502 for execution. The term “storage medium” as used herein refers to any non-transitory media that store data and/or instructions that cause a machine to operate in a specific fashion. Such a medium may take many forms, including, but not limited to, non-volatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 508. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

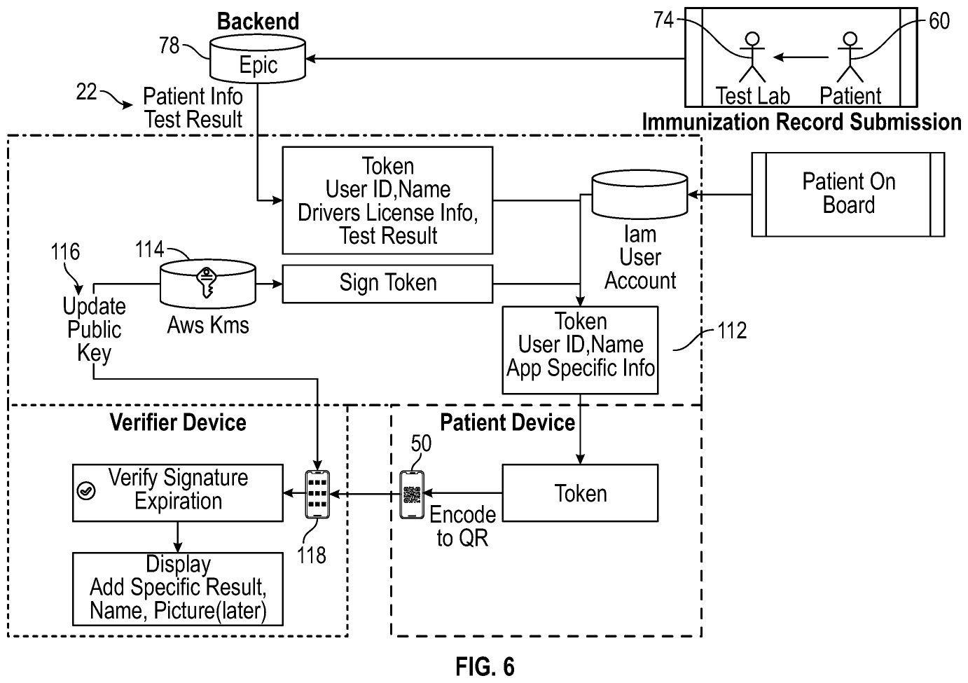

FIG. 6 is a diagram schematically illustrating alternative aspects of the exemplary secured validation system 10. As a non-limiting example, the patient or individual 60 registers through the validation service 140. In certain aspects, the individual 60 can register via the user device 50 to create the profile 62, which can include the profile data 64. In certain aspects, as part of the registration process, the individual 60 can upload an image of the individual 60 to the profile data 64. The image of the individual 60 can be an image such as, but not limited to, a driver's license, a passport, a government issued identification, a company issued identification, and other types of issued identifications that are well-known in the industry. The validation service 140 is configured to assign a universally unique identifier (UUID) to the image of the individual 60, which can be referenced later to verify the identity of the individual 60.

After the individual 60 is registered and has completed the test at the first entity 74, the validation service 140 receives the corresponding test result 22 from the first entity 74 via the at least one first entity device 72. In certain aspects, instead of receiving the test result 22 directly from the first entity 74, the validation service 140 receives the test result 22 from the second entity 78 via the at least one second entity device 76, which received the test result 22 from the first entity via the at least one first entity device 72. Once the validation service 140 receives the test result 22, the validation service 140 creates or generates a digital certificate or token 112 that includes the indication 86 of the test result 22 and the profile data 64. In certain aspects, in addition to the indication 86 of the test result 22 and the profile data 64, the token 112 also includes the UUID of the image of the individual 60.

In certain aspects, the validation service 140 communicates with a cryptographic key service 114, which digitally signs the token 112 for cryptographical protection from tampering of the token 112. In certain aspects, a public key 116 associated with the token 112 is generated. In certain aspects, the token 112 is signed using any crypto algorithm well-known in the industry such as, but not limited to, an Elliptic Curve Digital Signature Algorithm (ECDSA) with at least p256 curve (asa secp256r1) keys. The validation service 140 receives the digitally signed token 112 from the cryptographic key service 114 and creates the unique code 82 (e.g., QR code) associated with the digitally signed token 112 for transmitting to the user device 50. In certain aspects, the unique code 82 includes the digitally signed token 112, which can include, but is not limited to, the name of the individual 60, the UUID of the image of the individual 60, the indication 86 of the test result 22, the digital signature, and other information associated with the individual 60.

The individual 60 can display the unique code 82 on the user device 50 when the individual wishes to enter the establishment 84, which requires guests to have taken a test and have test results 22 that meet the criteria to allow the individual 60 to enter. The establishment 84 can then scan the unique code 82 via an establishment device 118 associated with the establishment 84 to verify whether the individual 60 meets the criteria for entry. The establishment device 118 can communicate with the validation service 140 to receive the public key 116. In certain aspects, the public key 116 supports key rotation such that the indication 86 associated with the digitally signed token 112 is updated when key rotation occurs. For example, the establishment device 118 can periodically communicate with the validation service 140 to receive the most up to date version of the public key 116. In certain aspects, two or more active public keys 116 are in rotation. In certain aspects, the public key 116 is configured to support certificate pinning such that the establishment device 118 with the public key 116 is pinned to the validation server 20 hosting the validation service 140. In certain aspects, the establishment device 118 is notified when the public key 116 is about to expire so that the establishment device 118 can actively request the most up to date version of the public key 116. The establishment device 118 verifies the scan of the unique code 82 against the public key 116 to ensure that the digitally signed token 112 has not be altered.

It should be understood that the disclosed system 10 provides improved scalability as the establishment device 118 will have the public key 116 and will not need to communicate with the validation service 140 for each verification scan. For example, such scalability provides improvements at large events where a surge of many verifications will be requested. The disclosed system 10 also provides privacy as the storing of medical records is not required.

In certain aspects, the validation service 140 creates a one-way hash using the token 112 and a nonce. In certain aspects, the one-way hash can be stored on the blockchain network 30. The one-way hash and the corresponding one-way hash that is stored on the blockchain network 30 can be used to verify the integrity of the digitally signed token 112 without having to store information other than the one-way hash on the blockchain network 30.

In one aspect, a method may be an operation, an instruction, or a function and vice versa. In one aspect, a clause or a claim may be amended to include some or all of the words (e.g., instructions, operations, functions, or components) recited in other one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A secured validation system, comprising:
a memory; and
a processor configured to execute instructions which, when executed, cause the processor to:
receive an enrollment request from a user device associated with an individual;
generate a profile associated with the individual, wherein the profile comprises profile data;
store the profile on a blockchain network;
receive a test result associated with a test of the individual;
append the profile of the individual with the test result;
determine whether a facility associated with the test result is approved certifying facility;
create, responsive to determining that the facility is one of the approved certifying facilities, a unique code associated with the individual, wherein the unique code comprises an indication of the test result; and
transmit the unique code to the user device associated with the individual.

2. The secured validation system of claim 1, wherein the test result associated with the test of the individual is received from a lab system entity.

3. The secured validation system of claim 1, wherein the test result associated with the test of the individual is received from a health system entity.

4. The secured validation system of claim 1, wherein the profile data comprises an image of the individual, and the processor is further configured to execute instructions which, when executed, cause the processor to assign a universally unique identifier to the image of the individual.

5. The secured validation system of claim 4, wherein the unique code is a digitally signed token comprising the indication of the test result and the profile data, wherein the digitally signed token is associated with a public key.

6. The secured validation system of claim 5, wherein the processor is further configured to execute instructions which, when executed, cause the processor to transmit, responsive to a request from an establishment device, the public key to the establishment device.

7. The secured validation system of claim 5, wherein the digitally signed token is signed using an Elliptic Curve Digital Signature Algorithm.

8. A computer-implemented method for securely validating a test, comprising:
receiving an enrollment request from a user device associated with an individual;
generating a profile associated with the individual, wherein the profile comprises profile data;
storing the profile on a blockchain network;
receiving a test result associated with a test of the individual;
updating the profile of the individual with the test result;
determining whether a facility associated with the test result is approved certifying facility;
creating, responsive to determining that the facility is one of the approved certifying facilities, a unique code associated with the individual, wherein the unique code comprises an indication of the test result;
transmitting the unique code to the user device associated with the individual.

9. The computer-implemented method of claim 8, wherein the profile data comprises an image of the individual, and the processor is further configured to execute instructions which, when executed, cause the processor to assign a universally unique identifier to the image of the individual.

10. The computer-implemented method of claim 9, wherein the unique code is a digitally signed token comprising the indication of the test result and the profile data, wherein the digitally signed token is associated with a public key.

11. The computer-implemented method of claim 10, wherein the processor is further configured to execute instructions which, when executed, cause the processor to transmit, responsive to a request from an establishment device, the public key to the establishment device.

12. The computer-implemented method of claim 10, wherein the public key supports key rotation.

13. The computer-implemented method of claim 11, wherein two or more active public keys are in key rotation.

14. The computer-implemented method of claim 10, wherein the public key is configured to support certificate pinning.

15. The computer-implemented method of claim 10, wherein the digitally signed token is signed using an Elliptic Curve Digital Signature Algorithm.

16. The computer-implemented method of claim 10, further comprises:
creating a one-way hash using the digitally signed token; and
storing the one-way hash on the blockchain network.

17. A non-transitory machine-readable storage medium comprising machine-readable instructions for causing a processor to execute a method for securely validating a test, the method comprising:
receiving an enrollment request from a user device associated with an individual;
generating a profile associated with the individual, wherein the profile comprises profile data;
storing the profile on a blockchain network;
receiving a test result associated with a test of the individual;
updating the profile of the individual with the test result;
determining whether a facility associated with the test result is approved certifying facility;
creating, responsive to determining that the facility is one of the approved certifying facilities, a unique code associated with the individual, wherein the unique code comprises an indication of the test result; and
transmitting the unique code to the user device associated with the individual.

18. The non-transitory machine-readable storage medium of claim 17, wherein the profile data comprises an image of the individual, and the processor further executes instructions to assign a universally unique identifier to the image of the individual.

19. The non-transitory machine-readable storage medium of claim 18, wherein the unique code is a digitally signed token comprising the indication of the test result and the profile data, wherein the digitally signed token is associated with a public key.

20. The non-transitory machine-readable storage medium of claim 19, wherein the processor further executes instructions to transmit, responsive to a request from an establishment device, the public key to the establishment device
